(19) United States
(12) Patent Application Publication
(10) Pub. No.: US 2005/0193444 A1
(43) Pub. Date: Sep. 1, 2005

(54) Transgenic corn seed with enhanced amino acid content

(76) Inventors: Thomas M. Malvar, Stonington, CT (US); Shihshieh Huang, Stonington, CT (US); Michael H. Luethy, Webster Groves, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 11/057,069

(22) Filed: Feb. 10, 2005

Related U.S. Application Data
(60) Provisional application No. 60/543,157, filed on Feb. 10, 2004.
(60) Provisional application No. 60/543,187, filed on Feb. 10, 2004.
(60) Provisional application No. 60/600,859, filed on Aug. 11, 2004.

(51) Int. Cl. A01H 1/00; C12N 15/82; A01H 5/00

(52) U.S. Cl. 800/278; 800/320.1

(57) ABSTRACT

Anti-sense-oriented RNA gene suppression agents in the form of a loop of anti-sense-oriented RNA is produced in cells of transgenic organisms, e.g. plants, by transcription from a recombinant DNA construct which comprises in 5′ to 3′ order a promoter element operably linked to an anti-sense-oriented DNA element and a complementary DNA element.

### CROSS REFERENCE TO RELATED APPLICATIONS

This application claims priority under 35 U.S.C. 119(e) to provisional applications Ser. No. 60/543,157, filed Feb. 10, 2004, No. 60/543,187, filed Feb. 10, 2004 and No. 60/600,859, filed Aug. 11, 2004, the disclosures of all of which are incorporated herein by reference in their entireties.

### INCORPORATION OF SEQUENCE LISTING

A computer readable form of the sequence listing is contained in the file named “53490.ST25.txt” which is 10.7 kb (measured in MS-Windows Explorer) and was created on Feb. 9, 2005 and is located on a CDROM, which is filed herewith and herein incorporated by reference.

### FIELD OF THE INVENTION

Disclosed herein are seeds for transgenic corn having elevated amino acid level, recombinant DNA constructs for producing gene-suppressing loops of anti-sense RNA and methods of making and using such constructs and transgenic plants expressing gene-suppressing loops of anti-sense RNA.

### BACKGROUND

Certain plants have low levels of specific amino acids compared to other plants, e.g. corn has low levels of lysine, methionine and tryptophan. Efforts to increase amino acid levels in transgenic plants include expressing recombinant DNA which encodes proteins in an amino acid synthesis pathway at higher levels than native genes. One such gene for producing enhanced levels of lysine in corn is a bacterial dihydropicolinic acid synthase as disclosed in U.S. Pat. Nos. 5,288,300 (Glassman et al.), 6,459,019 (Falco et al.) and Patent Application Publication U.S. 2003/0056242 A1, each of which is incorporated herein by reference in their entirety. A concept for even more enhanced levels of amino acids includes suppression of genes encoding proteins in amino acid catabolic pathways.

Gene suppression includes any of the well-known methods for suppressing transcription of a gene or the accumulation of the mRNA corresponding to that gene thereby preventing translation of the transcript into protein. More particularly, gene suppression mediated by inserting a recombinant DNA construct with anti-sense oriented DNA to regulate gene expression in plant cells is disclosed in U.S. Pat. No. 5,107,065 (Shewmaker et al.) and U.S. Pat. No. 5,759,829 (Shewmaker et al.). Plants transformed using such anti-sense oriented DNA constructs for gene suppression can comprise integrated DNA arranged as an inverted repeat that resulted from co-insertion of several copies of the transfer DNA (T-DNA) into plants by Agrobacterium-mediated transformation, as disclosed by Redenbaugh et al. in “Safety Assessment of Genetically Engineered Flavr Savr™ Tomato, CRC Press, Inc. (1992). Inverted repeat insertions can comprise a part or all of the T-DNA, e.g. contain an inverted repeat of a complete or partial anti-sense construct. Screening for inserted DNA comprising inverted repeat elements can improve the efficiency of identifying transformation events effective for gene silencing when the transformation construct is a simple anti-sense DNA construct.

Gene suppression triggered by inserting a recombinant DNA construct with sense-oriented DNA to regulate gene expression in plants is disclosed in U.S. Pat. No. 5,283,184 (Jorgensen et al.) and U.S. Pat. No. 5,231,020 (Jorgensen et al.). Inserted T-DNA providing gene suppression in plants transformed with such sense constructs by Agrobacterium is organized predominately in inverted repeat structures, as disclosed by Jorgensen et al., Mol. Gen. Genet., 207: 471-477 (1987). See also Stam et al., The Plant Journal, 12: 63-82 (1997) and De Buck et al., Plant Mol. Biol. 46 433-445 (2001), who used segregation studies to support Jorgensen's finding that in many events gene silencing is mediated by multimeric transgene T-DNA where the T-DNAs are arranged in inverted repeats. Screening for inserted DNA comprising inverted repeat elements can improve the gene silencing efficiency when transforming with simple sense-orientated DNA constructs.

Gene silencing can also be effected by transcribing RNA from both a sense and an anti-sense oriented DNA using two separate transcription units, e.g. as disclosed by Shewmaker et al. in U.S. Pat. No. 5,107,065 where in Example 1 a binary vector was prepared with both sense and anti-sense aroA genes. Similar constructs are disclosed in International Publication No. WO 99/53050 (Waterhouse et al.). See also U.S. Pat. No. 6,326,193 where gene targeted DNA is operably linked to opposing promoters.

Gene suppression can be achieved in plants by providing transformation constructs that are capable of generating an RNA that can form double-stranded RNA along at least part of its length. Gene suppression in plants is disclosed in EP 0426195 A1 (Goldbach et al.) where recombinant DNA constructs for transcription into hairpin RNA provided transgenic plants with resistance to tobacco spotted wilt virus. See also Sijen et al., The Plant Cell, Vol. 8, 2277-2294 (1996) which discloses the use of constructs carrying inverted repeats (sense followed by anti-sense) of a cowpea mosaic virus gene in transgenic plants to mediate virus resistance. See also International Publication No. 98/53083 (Grierson et al.) and related U.S. Patent Application Publication No. 2003/0175965 A1 (Lowe et al.) which disclose gene suppression, using a double stranded RNA construct comprising a gene coding sequence preceded by an inverted repeat of 5′UTR. Constructs for posttranscriptional gene suppression in plants by double-stranded RNA of the target gene are also disclosed in International Publication No. WO 99/53050 (Waterhouse et al.) and International Publication No. WO 99/49029 (Graham et al.). See also U.S. Patent Application Publication No. 2002/0048814 A1 (Oeller) where DNA constructs are transcribed to sense or anti-sense RNA with a hairpin-forming poly(T)-poly(A) tail. See also U.S. Patent Application Publication No. 2003/0018993 A1 (Gutterson et al.) where sense or anti-sense DNA is followed by an inverted repeat of the 3′ untranslated region of the NOS gene. See also U.S. Patent Application Publication No. 2003/0036197 A1 (Glassman et al.) where RNA for reducing the expression of target mRNA comprises a part with homology to target mRNA and a part with complementary RNA regions that are unrelated to endogenous RNA.

The production of dsRNA in plants to inhibit gene expression, e.g. in a nematode feeding on the plant, is disclosed U.S. Pat. No. 6,506,559 (Fire et al.). Multi-gene suppression vectors for use in plants are disclosed in U.S. patent application Ser. No. 10/465,800 (Fillatti).

Transcriptional suppression such as promoter trans suppression can be affected by a expressing a DNA construct comprising a promoter operably linked to inverted repeats of promoter DNA from a target gene. Constructs useful for such gene suppression mediated by promoter trans suppression are disclosed by Mette et al., The EMBO Journal, Vol. 18, pp. 241-148, (1999) and by Mette et al., The EMBO Journal, Vol. 19, pp. 5194-5201-148, (2000), both of which are incorporated herein by reference.

All of the above-described patents, applications and international publications disclosing materials and methods for gene suppression in plants are incorporated herein by reference.

### SUMMARY OF THE INVENTION

This invention provides seed for transgenic corn having enhanced amino acid content. Such transgenic corn with elevated amino acid in its kernels has integrated into its genome a recombinant DNA construct that transcribes an anti-sense-oriented RNA that suppresses the production of a protein in an amino acid catabolic pathway.

In one aspect of the invention the seed has recombinant DNA for suppressing a gene encoding a protein in a lysine catabolic pathway, e.g. the pre-polymer lysine ketoglutarate reductase/saccharopine dehydrogenase. A useful protein target for suppression is ketoglutarate reductase. Enhanced amino acid content can also be achieved by concurrently expressing a gene in an amino acid synthesis pathway, e.g. an exogenous gene coding for dihydrodipicolinate synthase in the lysine synthesis pathway. Thus, this invention also provides seeds and methods in which recombinant DNA is used to suppress a protein in an amino acid catabolic pathway and express, e.g. over express a protein in an amino acid synthesis pathway.

Another aspect of the invention provides methods of increasing the content of an amino acid, e.g. the lysine content in corn kernels, by expressing in developing corn seed a recombinant DNA construct for suppressing the expression of a protein in an amino acid catabolic pathway, and optionally, expressing a protein in an amino acid synthesis pathway.

The recombinant DNA constructs of this invention comprise an anti-sense-oriented DNA element from a gene targeted for suppression. The constructs also comprises sense-oreinted DNA that transcribes RNA that is complementary to at least part of the anti-sense-oriented RNA. In a preferred aspect of the recombinant DNA constructs of this invention the sense-oriented DNA element that is shorter than the anti-sense-oriented DNA element and sense-oriented RNA transcribed from the sense-oriented DNA element is complementary to the 5′-most part of anti-sense-oriented RNA transcribed from the anti-sense-oriented DNA element. Such transcribed RNA forms into a loop of anti-sense-oriented RNA for suppressing at least one target gene for a protein in an amino acid catabolic pathway.

Recombinant DNA constructs comprise a promoter, e.g. a seed specific promoter, operably linked to the DNA that is transcribed to the anti-sense-oriented RNA, e.g. that forms a loop of anti-sense-oriented RNA. Such recombinant DNA is useful for producing corn seed having an elevated amino acid content as compared to progeny seed from control corn plants in which production of a protein in the amino acid catabolic pathway is not suppressed, e.g. a wild type ancestor corn plant, or the negative segregant of the transgenic corn plant.

In preferred aspects of the invention the seed specific promoter is an embryo specific promoter or an endosperm specific promoter and the recombinant DNA construct produces anti-sense-oriented RNA for suppressing a gene encoding a protein in the lysine catabolic pathway, e.g. lysine ketoglutarate reductase and/or saccharopine dehydrogenase. In another aspect of the invention amino acid content is enhanced in transgenic corn further having integrated into its genome recombinant DNA which expresses a protein in an amino acid synthesis pathway, e.g. dihydropicolinate synthase. Thus, a unique aspect of this invention provides transgenic seeds and methods using a recombinant DNA construct for producing in a plant a loop of anti-sense-oriented RNA for gene suppression of lysine ketoglutarate reductase and/or saccharopine dehydogenase as well as for expressing an exogenous gene coding for dihydrodipicolinate synthase. Such constructs comprise in 5′ to 3′ order a seed specific promoter element operably linked to an anti-sense-oriented DNA element and sense-oriented DNA element from the gene coding for the preprotein lysine ketoglutarate reductase/saccharopine dehydrogenase. The sense-oriented DNA element is shorter than the anti-sense-oriented DNA and sense-oriented RNA transcribed by the sense-oriented DNA element is complementary to a 5′-most segment of anti-sense-oriented RNA transcribed by the anti-sense-oriented DNA element. The DNA elements are transcribed as RNA that forms into a loop of anti-sense-oriented RNA for suppressing the expression of the native gene coding for lysine ketoglutarate reductase.

### BRIEF DESCRIPTION OF THE DRAWING

FIG. 1 is a schematic illustration of a recombinant DNA construct useful in this invention to produce an anti-sense-oriented loop of RNA.

FIG. 2 is a Western analysis indicating gene suppression using a construct of this invention.

### DETAILED DESCRIPTION

SEQ ID NO: 1 is a nucleotide sequence of a recombinant DNA construct useful for transcribing RNA that can form an anti-sense-oriented RNA loop for suppressing one or multiple genes in transgenic plants. See Table 1 for a description of elements.

As used herein, “complementary” refers to polynucleotides that are capable of hybridizing, e.g. sense and anti-sense strands of DNA or self-complementary strands of RNA, due to complementarity of aligned nucleotides permitting C-G and A-T or A-U bonding.

As used herein “vector” means a DNA molecule capable of replication in a host cell and/or to which another DNA segment can be operatively linked so as to bring about replication of the attached segment. A plasmid is an exemplary vector.

As used herein a “transgenic” organism, e.g. plant or seed, is one whose genome has been altered by the incorporation of recombinant DNA comprising exogenous genetic material or additional copies of native genetic material, e.g. by transformation or recombination of the organism or an ancestral organism. Transgenic plants include progeny plants of an original plant derived from a transformation process including progeny of breeding transgenic plants with wild type plants or other transgenic plants. Crop plants of particular interest in the present invention include, but are not limited to maize, soybean, cotton, canola (rape), wheat, rice, sunflower, safflower and flax. Other crops of interest include plants producing vegetables, fruit, grass and wood.

### Recombinant DNA Constructs for Plant Transformation

Recombinant DNA constructs for producing looped, anti-sense RNA, gene suppression agents in transgenic plants can be readily prepared by those skilled in the art. Typically, such a DNA construct comprises as a minimum a promoter active in the tissue targeted for suppression, a transcribable DNA element having a sequence that is complementary to nucleotide sequence of a gene targeted for suppression and a transcription terminator element. The targeted gene element copied for use in transcribable DNA in the gene suppression construct can be a promoter element, an intron element, an exon element, a 5′ UTR element, or a 3′ UTR element. Although the minimum size of DNA copied from sequence of a gene targeted for suppression is believed to be about 21 or 23 nucleotides; larger nucleotide segments are preferred, e.g. up the full length of a targeted gene. The DNA element can comprise multiple parts of a gene, e.g. nucleotides that are complementary to contiguous or separated gene elements of UTR, exon and intron. Such constructs may also comprise other regulatory elements, DNA encoding transit peptides, signal peptides, selective markers and screenable markers as desired. To form an anti-sense-oriented RNA loop the complementary DNA element is conveniently not more than about one-half the length of the anti-sense-oriented DNA element, often not more than one-third the length of said anti-sense-oriented DNA element, e.g. not more than one-quarter the length of said anti-sense-oriented DNA element. The overall lengths of the combined DNA elements can vary. For instance, the anti-sense-oriented DNA element can consist of from 500 to 5000 nucleotides and the complementary DNA element can consist of from 50 to 500 nucleotides.

The anti-sense transcription unit can be designed to suppress multiple genes where the DNA is arranged with two or more anti-sense-oriented elements from different genes targeted for suppression followed by a complementary sense-oriented element, e.g. complementary to at least a part of the 5′ most anti-sense element.

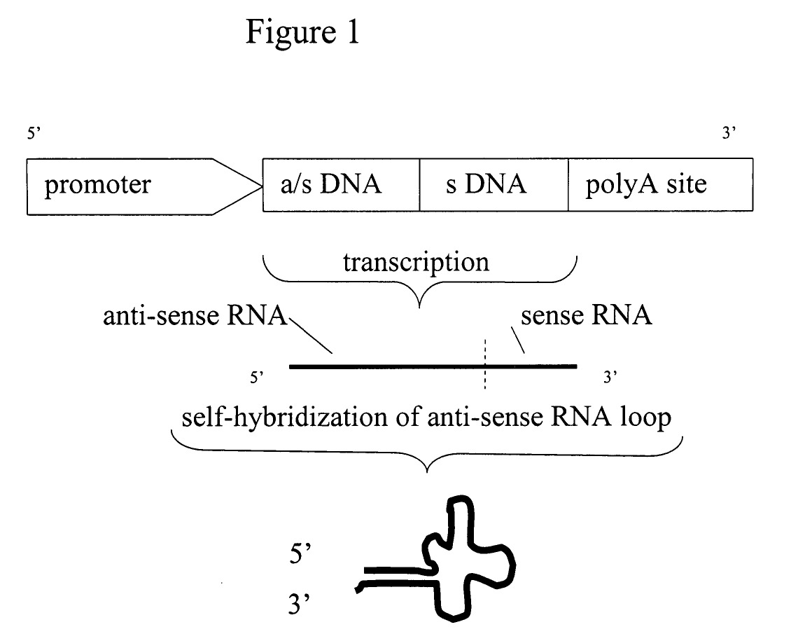

With reference to FIG. 1 there is schematically shown a recombinant DNA construct comprising a promoter element, an anti-sense-oriented DNA element (denoted “a/s DNA”), a complementary sense-oriented DNA element (denoted “s DNA”) and DNA providing polyadenylation signals and site (denoted “polyA site”). The DNA construct is transcribed to RNA comprising an anti-sense-oriented RNA segment and a complementary RNA segment which is complementary to the 5′-most end of the anti-sense-oriented RNA segment. The 5′ and 3′ ends of the anti-sense RNA can self hybridize to form a double-stranded RNA segment that closes a loop of anti-sense-oriented RNA. For example, if the nucleotide sequence of the 5′-most end of the strand of transcribed anti-sense-oriented DNA is 5′-CGGCATA—, the sequence of the 3′-most end of the transcribed strand of the inverted repeat DNA will be —TATGCCG-3′ which is readily cloned from the source DNA providing the anti-sense element. With such sequences the loop of anti-sense-oriented RNA will extend from one side of a dsRNA segment, e.g.

5′-GCCGUAU--------
3′-CGGCAUA--------

The anti-sense-oriented DNA and its self-complementary DNA can be contiguous or separated by vector DNA, e.g. up to about 100 nucleotides or so of vector DNA separating restriction sites used for vector assembly.

Recombinant DNA constructs can be assembled using commercially available materials and methods known to those of ordinary skill in the art. A useful technology for building DNA constructs and vectors for transformation is the GATEWAY™ cloning technology (available from Invitrogen Life Technologies, Carlsbad, Calif.) uses the site specific recombinase LR cloning reaction of the Integrase att system from bacterophage lambda vector construction, instead of restriction endonucleases and ligases. The LR cloning reaction is disclosed in U.S. Pat. Nos. 5,888,732 and 6,277,608, U.S. Patent Application Publications 2001283529, 2001282319 and 20020007051, all of which are incorporated herein by reference. The GATEWAY™ Cloning Technology Instruction Manual which is also supplied by Invitrogen also provides concise directions for routine cloning of any desired DNA into a vector comprising operable plant expression elements.

An alternative vector fabrication method employs ligation-independent cloning as disclosed by Aslanidis, C. et al., Nucleic Acids Res., 18, 6069-6074, 1990 and Rashtchian, A. et al., Biochem., 206, 91-97,1992 where a DNA fragment with single-stranded 5′ and 3′ ends are ligated into a desired vector which can then be amplified in vivo.

Numerous promoters that are active in plant cells have been described in the literature. These include promoters present in plant genomes as well as promoters from other sources, including nopaline synthase (nos) promoter and octopine synthase (ocs) promoters carried on tumor-inducing plasmids of Agrobacterium tumefaciens, caulimovirus promoters such as the cauliflower mosaic virus or figwort mosaic virus promoters. For instance, see U.S. Pat. Nos. 5,322,938 and 5,858,742 which disclose versions of the constitutive promoter derived from cauliflower mosaic virus (CaMV35S), U.S. Pat. No. 5,378,619 which discloses a Figwort Mosaic Virus (FMV) 35S promoter, U.S. Pat. No. 5,420,034 which discloses a napin promoter, U.S. Pat. No. 6,437,217 which discloses a maize RS81 promoter, U.S. Pat. No. 5,641,876 which discloses a rice actin promoter, U.S. Pat. No. 6,426,446 which discloses a maize RS324 promoter, U.S. Pat. No. 6,429,362 which discloses a maize PR-1 promoter, U.S. Pat. No. 6,232,526 which discloses a maize A3 promoter, U.S. Pat. No. 6,177,611 which discloses constitutive maize promoters, U.S. Pat. No. 6,433,252 which discloses a maize L3 oleosin promoter, U.S. Pat. No. 6,429,357 which discloses a rice actin 2 promoter and intron, U.S. Pat. No. 5,837,848 which discloses a root specific promoter, U.S. Pat. No. 6,084,089 which discloses cold inducible promoters, U.S. Pat. No. 6,294,714 which discloses light inducible promoters, U.S. Pat. No. 6,140,078 which discloses salt inducible promoters, U.S. Pat. No. 6,252,138 which discloses pathogen inducible promoters, U.S. Pat. No. 6,175,060 which discloses phosphorus deficiency inducible promoters, U.S. Pat. No. 6,635,806 which discloses a coixin promoter, U.S. 2002/0192813A1 which discloses 5′, 3′ and intron elements useful in the design of effective plant expression vectors, U.S. 2004/0216189 A1 which discloses a maize chloroplast aldolase promoter, and U.S. 2004/0123347A1 which discloses water-deficit inducible promoters, all of which are incorporated herein by reference. These and numerous other promoters that function in plant cells are known to those skilled in the art and available for use in recombinant polynucleotides of the present invention to provide for expression of desired genes in transgenic plant cells.

Furthermore, the promoters may be altered to contain multiple “enhancer sequences” to assist in elevating gene expression. Such enhancers are known in the art. By including an enhancer sequence with such constructs, the expression of the selected protein may be enhanced. These enhancers often are found 5′ to the start of transcription in a promoter that functions in eukaryotic cells, but can often be inserted upstream (5′) or downstream (3′) to the coding sequence. In some instances, these 5′ enhancing elements are introns. Particularly useful as enhancers are the 5′ introns of the rice actin 1 (see U.S. Pat. No. 5,641,876) and rice actin 2 genes, the maize alcohol dehydrogenase gene intron, the maize heat shock protein 70 gene intron (U.S. Pat. No. 5,593,874) and the maize shrunken 1 gene.

In other aspects of the invention, sufficient expression in plant seed tissues is desired to effect improvements in seed composition. Exemplary promoters for use for seed composition modification include promoters from seed genes such as napin (U.S. Pat. No. 5,420,034), maize L3 oleosin (U.S. Pat. No. 6,433,252), zein Z27 (Russell et al. (1997) Transgenic Res. 6(2): 157-166), globulin 1 (Belanger et al (1991) Genetics 129:863-872), glutelin 1 (Russell (1997) supra), and peroxiredoxin antioxidant (PerI) (Stacy et al. (1996) Plant Mol Biol. 31(6): 1205-1216).

Recombinant DNA constructs prepared in accordance with the invention will often include a 3′ element that typically contains a polyadenylation signal and site, especially if the recombinant DNA is intended for protein expression as well as gene suppression. Well-known 3′ elements include those from Agrobacterium tumefaciens genes such as nos 3′, tml 3′, tmr 3′, tms 3, ocs 3′, tr7 3′, e.g. disclosed in U.S. Pat. No. 6,090,627, incorporated herein by reference; 3′ elements from plant genes such as wheat (Triticum aesevitum) heat shock protein 17 (Hsp 17 3′), a wheat ubiquitin gene, a wheat fructose-1,6-biphosphatase gene, a rice glutelin gene a rice lactate dehydrogenase gene and a rice beta-tubulin gene, all of which are disclosed in U.S. published patent application 2002/0192813 A1, incorporated herein by reference; and the pea (Pisum sativum) ribulose biphosphate carboxylase gene (rbs 3′), and 3′ elements from the genes within the host plant.

The gene-suppressing recombinant DNA construct can also be stacked with DNA imparting other traits of agronomic interest including DNA providing herbicide resistance or insect resistance such as using a gene from Bacillus thuringensis to provide resistance against lepidopteran, coliopteran, homopteran, hemiopteran, and other insects. Herbicides for which resistance is useful in a plant include glyphosate herbicides, phosphinothricin herbicides, oxynil herbicides, imidazolinone herbicides, dinitroaniline herbicides, pyridine herbicides, sulfonylurea herbicides, bialaphos herbicides, sulfonamide herbicides and glufosinate herbicides. Persons of ordinary skill in the art are enabled in providing stacked traits by reference to U.S. patent application publications 2003/0106096A1 and 2002/0112260A1 and U.S. Pat. Nos. 5,034,322; 5,776,760; 6,107,549 and 6,376,754 and to insect/nematode/virus resistance by reference to U.S. Pat. Nos. 5,250,515; 5,880,275; 6,506,599; 5,986,175 and U.S. Patent Application Publication 2003/0150017 A1, all of which are incorporated herein by reference.

Transformation Methods—Numerous methods for transforming plant cells with recombinant DNA are known in the art and may be used in the present invention. Two commonly used methods for plant transformation are Agrobacterium-mediated transformation and microprojectile bombardment. Microprojectile bombardment methods are illustrated in U.S. Pat. Nos. 5,015,580 (soybean); 5,550,318 (corn); 5,538,880 (corn); 5,914,451 (soybean); 6,160,208 (corn); 6,399,861 (corn) and 6,153,812 (wheat) and Agrobacterium-mediated transformation is described in U.S. Pat. Nos. 5,159,135 (cotton); 5,824,877 (soybean); 5,591,616 (corn); and 6,384,301 (soybean), all of which are incorporated herein by reference. For Agrobacterium tumefaciens based plant transformation system, additional elements present on transformation constructs will include T-DNA left and right border sequences to facilitate incorporation of the recombinant polynucleotide into the plant genome.

In general it is useful to introduce recombinant DNA randomly, i.e. at a non-specific location, in the genome of a target plant line. In special cases it may be useful to target recombinant DNA insertion in order to achieve site-specific integration, e.g. to replace an existing gene in the genome, to use an existing promoter in the plant genome, or to insert a recombinant polynucleotide at a predetermined site known to be active for gene expression. Several site specific recombination systems exist which are known to function implants include cre-lox as disclosed in U.S. Pat. No. 4,959,317 and FLP-FRT as disclosed in U.S. Pat. No. 5,527,695, both incorporated herein by reference.

Transformation methods of this invention are preferably practiced in tissue culture on media and in a controlled environment. “Media” refers to the numerous nutrient mixtures that are used to grow cells in vitro, that is, outside of the intact living organism. Recipient cell targets include, but are not limited to, meristem cells, callus, immature embryos and gametic cells such as microspores, pollen, sperm and egg cells. It is contemplated that any cell from which a fertile plant may be regenerated is useful as a recipient cell. Callus may be initiated from tissue sources including, but not limited to, immature embryos, seedling apical meristems, microspores and the like. Cells capable of proliferating as callus are also recipient cells for genetic transformation. Practical transformation methods and materials for making transgenic plants of this invention, e.g. various media and recipient target cells, transformation of immature embryos and subsequent regeneration of fertile transgenic plants are disclosed in U.S. Pat. Nos. 6,194,636 and 6,232,526, which are incorporated herein by reference.

The seeds of transgenic plants can be harvested from fertile transgenic plants and be used to grow progeny generations of transformed plants of this invention including hybrid plants line for screening of plants having an enhanced agronomic trait. In addition to direct transformation of a plant with a recombinant DNA, transgenic plants can be prepared by crossing a first plant having a recombinant DNA with a second plant lacking the DNA. For example, recombinant DNA can be introduced into first plant line that is amenable to transformation to produce a transgenic plant which can be crossed with a second plant line to introgress the recombinant DNA into the second plant line. A transgenic plant with recombinant DNA providing an enhanced agronomic trait, e.g. enhanced yield, can be crossed with transgenic plant line having other recombinant DNA that confers another trait, e.g. herbicide resistance or pest resistance, to produce progeny plants having recombinant DNA that confers both traits. Typically, in such breeding for combining traits the transgenic plant donating the additional trait is a male line and the transgenic plant carrying the base traits is the female line. The progeny of this cross will segregate such that some of the plants will carry the DNA for both parental traits and some will carry DNA for one parental trait; such plants can be identified by markers associated with parental recombinant DNA Progeny plants carrying DNA for both parental traits can be crossed back into the female parent line multiple times, e.g. usually 6 to 8 generations, to produce a progeny plant with substantially the same genotype as one original transgenic parental line but for the recombinant DNA of the other transgenic parental line

In the practice of transformation DNA is typically introduced into only a small percentage of target cells in any one transformation experiment. Marker genes are used to provide an efficient system for identification of those cells that are stably transformed by receiving and integrating a transgenic DNA construct into their genomes. Preferred marker genes provide selective markers which confer resistance to a selective agent, such as an antibiotic or herbicide. Any of the herbicides to which plants of this invention may be resistant are useful agents for selective markers. Potentially transformed cells are exposed to the selective agent. In the population of surviving cells will be those cells where, generally, the resistance-conferring gene is integrated and expressed at sufficient levels to permit cell survival. Cells may be tested further to confirm stable integration of the exogenous DNA. Commonly used selective marker genes include those conferring resistance to antibiotics such as kanamycin and paromomycin (nptII), hygromycin B (aph IV) and gentamycin (aac3 and aacC4) or resistance to herbicides such as glufosinate (bar orpat) and glyphosate (aroA or EPSPS). Examples of such selectable are illustrated in U.S. Pat. Nos. 5,550,318; 5,633,435; 5,780,708 and 6,118,047, all of which are incorporated herein by reference. Screenable markers which provide an ability to visually identify transformants can also be employed, e.g., a gene expressing a colored or fluorescent protein such as a luciferase or green fluorescent protein (GFP) or a gene expressing a beta-glucuronidase or uidA gene (GUS) for which various chromogenic substrates are known.

Cells that survive exposure to the selective agent, or cells that have been scored positive in a screening assay, may be cultured in regeneration media and allowed to mature into plants. Developing plantlets can be transferred to plant growth mix, and hardened off, e.g., in an environmentally controlled chamber at about 85% relative humidity, 600 ppm CO₂, and 25-250 microeinsteins m⁻² s⁻¹ of light, prior to transfer to a greenhouse or growth chamber for maturation. Plants are regenerated from about 6 weeks to 10 months after a transformant is identified, depending on the initial tissue. Plants may be pollinated using conventional plant breeding methods known to those of skill in the art and seed produced, e.g. self-pollination is commonly used with transgenic corn. The regenerated transformed plant or its progeny seed or plants can be tested for expression of the recombinant DNA and screened for the presence of enhanced agronomic trait.

### Transgenic Plants and Seeds

Transgenic plant seed provided by this invention are grown to generate transgenic plants having an enhanced trait as compared to a control plant. Such seed for plants with enhanced agronomic trait is identified by screening transformed plants or progeny seed for enhanced trait. For efficiency a screening program is designed to evaluate multiple transgenic plants (events) comprising the recombinant DNA, e.g. multiple plants from 2 to 20 or more transgenic events.

Transgenic plants grown from transgenic seed provided herein demonstrate improved agronomic traits that contribute to increased yield or other trait that provides increased plant value, including, for example, improved seed quality such as increased level of certain amino acids, e.g. lysine.

Many transgenic events which survive to fertile transgenic plants that produce seeds and progeny plants will not exhibit an enhanced agronomic trait. Screening is necessary to identify the transgenic plant having enhanced agronomic traits from populations of plants transformed as described herein by evaluating transgenic plants for the enhanced trait and minimal affect in other agronomic traits. These assays also may take many forms, including but not limited to, analyses to detect changes in the chemical composition, biomass, physiological properties, morphology of the plant.

The methods of this invention provide a means for a person of ordinary skill in the art to design recombinant DNA constructs, make transgenic plants, screen for enhanced amino acid level in seed and minimal adverse effect in other agronomic traits, to provide transgenic seed of this invention. Such seed can be used to produce a transgenic corn plant having integrated into its genome a recombinant DNA construct which transcribes anti-sense-oriented RNA that suppresses the level of a protein in an amino acid catabolic pathway.

The following examples illustrate aspects of the invention.

### EXAMPLE 1

This example illustrates preparation of a transformation vector useful for inserting a recombinant DNA construct of this invention into a transgenic plant to practice a method of this invention.

The LKR/SDH gene encodes a pre-protein for lysine ketoglutarate reductase (LKR) and saccharopine dehydrogenase (SDH) which are enzymes in a lysine catabolic pathway. Suppression of LKR is manifest in modification, e.g. increase, of lysine content. Suppression of LKR is effected by expressing in a plant a recombinant DNA construct that produces a stabilized anti-sense RNA transcribed from anti-sense-oriented LKR DNA and sense-oriented LKR DNA which forms a loop of anti-sense-oriented RNA. A transformation vector is prepared comprising two transcription units between right and left borders from Agrobacterium tumefaciens. One transcription unit for a marker comprised:

(a) DNA of a rice actin promoter and rice actin intron,

(b) DNA of a chloroplast transit peptide from Arabidopsis EPSPS

(c) DNA of A. tumefaciens aroA (a glyphosate-resistant marker), and

(d) DNA of A. tumefaciens NOS terminator,

The other transcription unit for LKR gene suppression comprised:

(a) DNA of Zea mays GLB1 promoter,

(b) DNA of a Zea mays ADH1 intron,

(c) Anti-sense-oriented DNA fragment of Zea mays LKR,

(d) Sense-oriented DNA fragment of Zea mays LKR, and

(e) DNA of Zea mays GLB1 terminator.

SEQ ID NO: 1 is a DNA sequence of a transformation vector comprising the above-described marker and gene suppression transcription units. See Table 1 below for a description of the elements of the transformation vector contained within SEQ ID NO: 1

TABLE 1

| Bases of SEQ | |
|---|---|
| ID NO: 1 | Description of DNA segment |
| 1-357 | A. tumefaciens right border |
| 376-1774 | DNA of a rice actin promoter and rice actin intron |
| 1784-2011 | DNA of A. tumefaciens EPSPS chloroplast transit |
| | peptide |
| 2012-3379 | DNA of A. tumefaciens aroA (glyphosate-resistant |
| | marker) |
| 3395-3647 | DNA of A. tumefaciens NOS terminator |
| 3691-4686 | DNA of Zea mays Glb1 terminator |
| 4692-5145 | Sense-oriented DNA element from Zea mays LKR |
| 5152-6118 | Anti-sense-oriented DNA element from Zea mays LKR |
| 6123-6680 | DNA of a Zea mays ADH1 intron |
| 6687-8082 | DNA of Zea mays GLB1 promoter |
| 8149-8590 | A. tumefaciens left border |

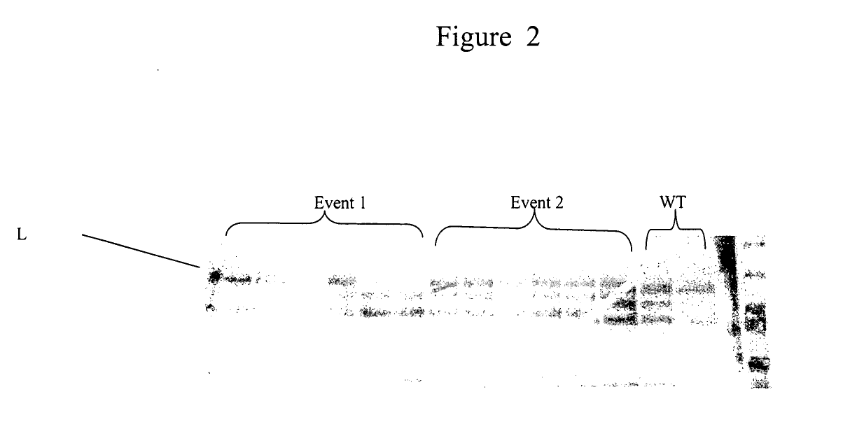

A vector prepared with the elements listed in Table 1 was used to transform corn plant tissue. Transgenic corn plants were obtained by Agrobacterium-mediated transformation. Transgenic plants from two separate transgenic insertion events were grown to produce F1 seed. Six mature seeds from each event were analyzed to determine success of transformation and suppression of LK-R. The mature transgenic seeds were dissected to extract protein which was analyzed by Western analysis. With reference to FIG. 2, seed from one of the events showed no reduction in LKAR as compared to wild type; and seed from the other event was shown to be segregating (1:1 hemizygous:wild type) as three of the six seeds showed substantial reduction in LKR as compared to wild type.

### EXAMPLE 2

This example illustrates transgenic corn with enhanced lysine. The transformation vector prepared in Example 1 is modified by inserting a transcription unit comprising a seed specific promoter operably linked to DNA coding for dihydrodipicolinate synthase. More specifically the transcription unit comprises DNA of a maize globulin 1 promoter (bp 48 to 1440; Kriz, Biochem. Genet. 27:239-251, 1989; Belanger and Kriz, Genetics, 129:863-872, 1991 and U.S. Pat. No. 6,329,574), a rice actin 1 intron (bp 1448 to 1928; McElroy et. al., Plant Cell, 2:163-171, 1990), a maize DHDPS chloroplast transit peptide (bp 1930 to 2100; Frisch et al., Mol. Gen. Genet., 228:287-293, 1991), a Corynebacterium DHDPS gene (bp 2101 to 3003; Bonnassie et al., Nucleic Acids Research, 18:6421, 1990; Richaud et al., J. Bacteriol., 166:297-300, 1986), a maize globulin 1 3′ untranslated region (bp 3080 to 4079; Belanger and Kriz, 1991). The promoters for the suppression of lysine ketoglutarate synthase and expression of dihidrodipicolinate synthase are adjacent to transcribe RNA in opposing directions. Corn produced from transgenic plants has higher levels of lysine, e.g. in the range of 3000 to 4000 ppm. as compared to essentially no lysine in type corn.

All of the materials and methods disclosed and claimed herein can be made and used without undue experimentation as instructed by the above disclosure. Although the materials and methods of this invention have been described in terms of preferred embodiments and illustrative examples, it will be apparent to those of skill in the art that variations may be applied to the materials and methods described herein without departing from the concept, spirit and scope of the invention. All such similar substitutes and modifications apparent to those skilled in the art are deemed to be within the spirit, scope and concept of the invention as defined by the appended claims.

1. Seed for producing a transgenic corn with enhanced amino acid content having integrated into its genome a recombinant DNA construct which transcribes anti-sense-oriented RNA that suppresses the production of a protein in an amino acid catabolic pathway, wherein the recombinant DNA comprises a seed specific promoter operably linked to DNA that is transcribed to said RNA and wherein said seed has an elevated amino acid content as compared to progeny seed from control corn plants in which production of said protein is not suppressed.

2. Seed according to claim 1 wherein said DNA that is transcribed to said RNA comprises an anti-sense-oriented DNA element and a sense-oriented DNA element, wherein the sense-oriented DNA element is shorter than the anti-sense-oriented DNA element, wherein sense-oriented RNA transcribed by the sense-oriented DNA is complementary to the 5′-most end of anti-sense-oriented RNA transcribed by the anti-sense-oriented DNA element, wherein said transcribed RNA forms a into a loop of anti-sense-oriented RNA for suppressing said protein in an amino acid catabolic pathway.

3. Seed according to claim 1 wherein said seed specific promoter is an embryo specific promoter or an endosperm specific promoter.

4. Seed according to claim 1 wherein said recombinant DNA construct produces RNA for suppressing a gene encoding a protein in the lysine catabolic pathway.

5. Seed according to claim 4 wherein said protein in the lysine catabolic pathway is lysine ketoglutarate reductase, saccharopine dehydrogenase or both.

6. Seed according to claim 1 further having integrated into its genome recombinant DNA which expresses a protein in an amino acid synthesis pathway.

7. Seed according to claim 6 wherein said protein in an amino acid synthesis pathway is dihydropicolinate synthase.

8. Seed according to claim 3 wherein said amino acid is lysine, said protein in an amino acid catabolic pathway is lysine ketoglutarate and said protein in an amino acid synthesis pathway is dihydropicolinate synthase.

9. A recombinant DNA construct for producing in a plant a loop of anti-sense-oriented RNA for gene suppression, wherein said construct comprises in 5′ to 3′ order a seed specific promoter element operably linked to an anti-sense-oriented DNA element and sense-oriented DNA element, wherein said sense-oriented DNA element is shorter than the ant-sense-oriented DNA element, wherein sense-oriented RNA transcribed by the sense-oriented DNA element is complementary to a 5′-most segment of anti-sense-oriented RNA transcribed by the anti-sense-oriented DNA element, wherein said DNA elements are transcribed as RNA that forms a into a loop of anti-sense-oriented RNA for suppressing the expression of at least one gene; wherein said gene targeted for suppression expresses lysine ketoglutarate reductase.

10. A method of increasing the level of lysine in corn seed by expressing a recombinant DNA construct of claim 9 in developing corn seed.

11. A method for producing corn seeds with enhanced amino acid level comprising growing corn plants from transgenic seed having integrated into its genome a recombinant DNA construct for suppressing the expression of a protein in an amino acid catabolic pathway, wherein said recombinant DNA construct comprises a seed specific promoter operably linked to DNA that is transcribed to anti-sense-oriented RNA complementary to messenger RNA for said protein and wherein said transgenic corn has an elevated amino acid content in its kernels as compared to a control corn plant in which said protein is not suppressed.

12. A method of claim 11 wherein said recombinant DNA construct comprises in 5′ to 3′ order said seed specific promoter operably linked to an anti-sense-oriented DNA element and sense-oriented DNA element, wherein said sense-oriented DNA element is shorter than said anti-sense-oriented DNA element, wherein sense-oriented RNA transcribed by the sense-oriented DNA element is complementary to a 5′-most segment of anti-sense-oriented RNA transcribed by the anti-sense-oriented DNA element, and wherein said DNA elements are transcribed as RNA that forms a into a loop of anti-sense-oriented RNA for suppressing the expression of said protein.

13. A method of claim 12 wherein said seed specific promoter is an embryo specific promoter or an endosperm specific promoter.

14. A method of claim 12 wherein said recombinant DNA construct produces RNA for suppressing the expression of a protein in the lysine catabolic pathway.

15. A method of claim 14 wherein said protein gene in the lysine catabolic pathway is lysine ketoglutarate reductase, saccharopine dehydrogenase or both.

16. A method of claim 12 further having integrated into its genome recombinant DNA which expresses a protein in an amino acid synthesis pathway.

17. A method of claim 16 wherein said protein in an amino acid synthesis pathway is dihydropicolinate synthase.

18. A method of claim 12 wherein said amino acid is lysine, said protein in an amino acid catabolic pathway is lysine ketoglutarate and said protein in an amino acid synthesis pathway is dihydropicolinate synthase.


SEQUENCE LISTING (1 sequence)

SEQ ID NO: 1 - DNA, 8296 bp, Artificial
  recombinant DNA construct in plasmid between Agrabacterium borders
ggtttacccgccaatatatcctgtcaaacactgatagtttaaactgaaggcgggaaacgacaatctgatccccatcaagcttactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccagcccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgtttttttttccgtctcggtctcgatctttggccttggtagtttgggtgggcgagaggcggcttcgtgccgcccagatcggtgcgcgggaggggcgggatctcgcggctggctctcgcccccgtggatccggcccggatctcgcggggaatggggctctcggatgtagatctgcgatccgccgttgttggggccgatgatggggcccttaaaatttccgccgtgctaaacaagatcaggaagaggggaaaagggcactatggtttatatttttatatatttctgctgcttcgtcaggcttagatgtgctagatctttctttcttctttttgtgggtagaatttaatccctcagcattgttcatcggtagtttttcttttcatgattcgtgacaaatgcagcctcgtgcggacgtttttttgtaggtagaagtgatcaaccatggcgcaagttagcagaatctgcaatggtgtgcagaacccatctcttatctccaatctctcgaaatccagtcaacgcaaatctcccttatcggtttctctgaagacgcagcagcatccacgagcttatccgatttcgtcgtcgtggggattgaagaagagtgggatgacgttaattggctctgagcttcgtcctcttaaggtcatgtcttctgtttccacggcgtgcatgcttcacggtgcaagcagccggcccgcaaccgcccgcaaatcctctggcctttccggaaccgtccgcattcccggcgacaagtcgatctcccaccggtccttcatgttcggcggtctcgcgagcggtgaaacgcgcatcaccggccttctggaaggcgaggacgtcatcaatacgggcaaggccatgcaggcgatgggcgcccgcatccgtaaggaaggcgacacctggatcatcgatggcgtcggcaatggcggcctcctggcgcctgaggcgccgctcgatttcggcaatgccgccacgggctgccgcctgacgatgggcctcgtcggggtctacgatttcgacagcaccttcatcggcgacgcctcgctcacaaagcgcccgatgggccgcgtgttgaacccgctgcgcgaaatgggcgtgcaggtgaaatcggaagacggtgaccgtcttcccgttaccttgcgcgggccgaagacgccgacgccgatcacctaccgcgtgccgatggcctccgcacaggtgaagtccgccgtgctgctcgccggcctcaacacgcccggcatcacgacggtcatcgagccgatcatgacgcgcgatcatacggaaaagatgctgcagggctttggcgccaaccttaccgtcgagacggatgcggacggcgtgcgcaccatccgcctggaaggccgcggcaagctcaccggccaagtcatcgacgtgccgggcgacccgtcctcgacggccttcccgctggttgcggccctgcttgttccgggctccgacgtcaccatcctcaacgtgctgatgaaccccacccgcaccggcctcatcctgacgctgcaggaaatgggcgccgacatcgaagtcatcaacccgcgccttgccggcggcgaagacgtggcggacctgcgcgttcgctcctccacgctgaagggcgtcacggtgccggaagaccgcgcgccttcgatgatcgacgaatatccgattctcgctgtcgccgccgccttcgcggaaggggcgaccgtgatgaacggtctggaagaactccgcgtcaaggaaagcgaccgcctctcggccgtcgccaatggcctcaagctcaatggcgtggattgcgatgagggcgagacgtcgctcgtcgtgcgtggccgccctgacggcaaggggctcggcaacgcctcgggcgccgccgtcgccacccatctcgatcaccgcatcgccatgagcttcctcgtcatgggcctcgtgtcggaaaaccctgtcacggtggacgatgccacgatgatcgccacgagcttcccggagttcatggacctgatggccgggctgggcgcgaagatcgaactctccgatacgaaggctgcctgatgagctcgaattcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggatgggggatccactagtgatatccgtcgagtggcggccgcgttttatgaataataataatgcatatctgtgcattactacctgggatacaagggcttctccgccataacaaattgagttgcgatgctgagaacgaacggggaagaaagtaagcgccgcccaaaaaaaacgaacatgtacgtcggctatagcaggtgaaagttcgtgcgccaatgaaaagggaacgatatgcgttgggtagttgggatacttaaatttggagagtttgttgcatacactaatccactaaagttgtctatctttttaacagctctaggcaggatataagatttatatctaatctgttggagttgcttttagagtaacttttctctctgtttcgtttatagccgattagcacaaaattaaactaggtgacgagaaataaagaaaaacggaggcagtaaaaaatacccaaaaaaatacttggagatttttgtctcaaaattatcttctaattttaaaagctacatattaaaaatactatatattaaaaatacttcgagatcattgcttgggatgggcagggccaatagctaattgctaaggatgggctatatttatgtatcgtctgaaacatgtaggggctaatagttagatgactaatttgctgtgttcgtacggggtgctgtttgagcctagcgatgaagggtcatagtttcatacaagaactcacttttggttcgtctgctgtgtctgttctcagcgtaacggcatcaatggatgccaaactccgcaaggggacaaatgaagaagcgaagagattatagaacacgcacgtgtcattatttatttatggacttgcctcagtagcttacagcatcgtacccgcacgtacatactacagagccacacttattgcactgcctgccgcttacgtacatagttaacacgcagagaggtatatacatacacgtccaacgtctccactcaggctcatgctacgtacgcacgtcggtcgcgcgccaccctctcgttgcttcctgctcgttttggcgaattccgatttggcaagtgttccagagcaaaagctggaagctctcgtagtctgagcctctttgctgattcatacaagttatgaccatctacatggatcgtctcaccaagaaatttgtagactgcaggatttttccctgaccggagtgcaccagctgggttccaactgaatttataggcaagcggattgtttgctgcagctggagatggcaatccaccacagtaagatgtaaatgcctttatttttccctttcgtgcatgagcttcatcaatcatcttcattgacatcaagtgatctatgccaggatctaggcccatttcacaaagtatagttacacctgcatctttggcagcttggctcaagtttgacatggattcatcaacatagcttgccgttaccatgtgcttcttcaactctatgcatactcctgcaatggcagcatgaaaactagcaggcagcaccggttggacatcattgagacagctggaggttcatttcacttggttagatgtgaagttggacaaagcacggatgatatgtcgtactcagagcttgaagtaggagcagatgatactgccacattggataaaattattgattccttgacttctttagctaatgaacatggtggagatcacgatgccgggcaagaaattgaattagctctgaagataggaaaagtcaatgagtatgaaactgacgtcacaattgataaaggagggccaaagattttaattcttggagctggaagagtctgtcggccagctgctgagtttctggcatcttacccagacatatgtacctatggtgttgatgaccatgatgcagatcaaattcatgttatcgtggcatctttgtatcaaaaagatgcagaagagacagttgatggtattgaaaatacaactgctacccagcttgatgttgctgatattggaagcctttcagatcttgtttctcaggttgaggttgtaattagcttgctgcctgctagttttcatgctgccattgcaggagtatgcatagagttgaagaagcacatggtaacggcaagctatgttgatgaatccatgtcaaacttgagccaagctgccaaagatgcaggtgtaactatactttgtgaaatgggcctagatcctggcatagatcacttgatgtcaatgaagatgattgatgaagctcatgcacgaaagggaaaaataaaggcatttacatcttactgtggtggattgccatctccagctgcagcaaacaatccgcttgcctataaattcagttggaacccagctggtgcactccggtcagggaaaaatcctgcagtctacaaatttcttggtgagacgatccatgtagatggtcataacttgtatgaatcagcaaagaggctcagactacgagagcttccagcttttgctctggaacacttgccaaatcgggatccgcagctgcacgggtccaggaaagcaatcgcatagtcaagctaaatcatcaagatgcaaacttttcgcccttgctaaacacggtaaaattcgaatggacatgtgtggagcagcaaaggccttacgtccgagaaacagggccactcaacgagttagttaaattcaaagaaagaaacgcctccttgcaagttgcaacattcttagatcatactgatgaaaatgacgtctttcattaaagaacagggaagatagatctttgctcaatatcgtatgatgtgttcagccagactgtcggatggaccacacggtaatagcagtgctggacgatgttacatcgagaaagattactagccttttcatgggagtgaaggatataaaagaaataagttcaccacgattgcaggatagcatacaagatcagcgccactgcggcactgttcatcgaaaaaaaaactgtggacgaagctagctttccccaaaattactcaacgaatcataaaccaagattagtcagatcaagagacagaggagaaacaaggcggacctttgcacttgatcggatccttgggttggctgtatgcagaactaaagcggaggtggcgcgcatttataccagcgccgggccctggtacgtggcgcggccgcgcggctacgtggaggaaggctgcgtggcagcagacacacgggtcgccacgtcccgccgtactctccttaccgtgcttatccgggctccggctcggtgcacgccagggtgtggccgcctctgagcagactttgtcgtgttccacagtggtgtcgtgttccggggactccgatccgcggcgagcgaccgagcgtgtaaaagagttcctactaggtacgttcattgtatctggacgacgggcagcggacaatttgctgtaagagaggggcagtttttttttagaaaaacagagaattccgttgagctaattgtaattcaacaaataagctattagttggttttagcttagattaaagaagctaacgactaatagctaataattagttggtctattagttgactcattttaaggccctgtttcaatctcgcgagataaactttagcagctattttttagctacttttagccatttgtaatctaaacaggagagctaatggtggtaattgaaactaaactttagcacttcaattcatatagctaaagtttagcaggaagctaaactttatcccgtgagattgaaacggggcctaaatctctcagctatttttgatgcaaattactgtcactactggaatcgagcgctttgccgagtgtcaaagcctgaaaaacactccgtaaagactttgcctagtgtgacactcgacaaagagatctcgacgaacagtacatcgacaacggcttctttgtcgagtactttttatcggacacttgacaaagtctttgtcgagtgaactacattgaaactctatgattttatgtgtaggtcacttaggtttctacacatagtacgtcacaactttaccgaaacattatcaaatttttatcacaacctctatatatgatatcatgacatgtggacaagtttcattaatttctgactttatttgtgttttatacaatttttaaacaactagataacaagttcacggtcatgtttagtgagcatggtgcttgaagattctggtctgcttctgaaatcggtcgtaacttgtgctagataacatgcatatcatttattttgcatgcacggttttccatgtttcgagtgacttgcagtttaaatgtgaattttccgaagaaattcaaataaacgaactaaatctaatatttatagaaaacatttttgtaaatatgtaattgtgccaaaatggtacatgtagatctacatagtgtaggaacataccacaaaaagtttggttggcaaaataaaaaaaataaaatatactttatccgagtgtccaaggtatggcactcggcccgggtggccaagcttactagcccgggcgcgccttaattaagcggccgcatcgatcgtgaagtttctcatctaagcccccatttggacgtgaatgtagacacgtcgaaataaagatttccgaattagaataatttgtttattgctttcgcctataaatacgacggatcgtaatttgtcgttttatcaaaatgtactttcattttataataacgctgcggacatctacatttttgaattgaaaaaaaattggtaattactctttctttttctccatattgaccatcatactcattgctgatccatgtagatttcccggacatgaagccatttacaattgaatatatcctgccgccgctgccgctttgcacccggtggagcttgcatgttggtttctacgcagaactgagccggttaggcagataatttccattgagaactgagccatgtgcaccttccccccaacacggtgagcgacggggcaacggagtgatccacatgggactttt